(19) United States
(12) Patent Application Publication
(10) Pub. No.: US 2022/0361761 A1
(43) Pub. Date: Nov. 17, 2022

(54) Method, a device, and a system for estimating a measure of cardiovascular health of a subject

(71) Applicant: Stichting IMEC Nederland, AE Eindhoven (NL)

(72) Inventors: Fabian BEUTEL, Duesseldorf (DE); Evelien HERMELING, Soerendonk (NL)

(21) Appl. No.: 17/240,200

(22) Filed: Apr. 26, 2021

(51) Int. Cl. A61B 5/02; A61B 5/0285; A61B 5/021; A61B 5/00; G16H 50/30; G16H 50/70; G16H 10/60; G16H 40/67

(52) U.S. Cl. CPC A61B 5/02007; A61B 5/0285; A61B 5/02125; A61B 5/7278; G16H 50/30; G16H 50/70; G16H 10/60; G16H 40/67

(57) ABSTRACT

A method for estimating a measure of cardiovascular health of a subject comprises: receiving (106) time-based sequences of at least a first and a second artery signal, each representative of pressure pulse wave propagation in an artery and representing pressure pulse wave propagation in positions displaced in relation to each other in the artery; fitting (110) a first and a second waveform to a portion of the time-based sequences to form a first and a second waveform of the first artery signal and a first and a second waveform of the second artery signal, wherein the first waveforms represent a forward propagating wave and the second waveforms represent a backward propagating wave; and determining (112) at least one parameter based on the fitting, wherein the at least one parameter comprises a forward velocity of the pressure pulse wave propagation as a representation of local pulse wave velocity in the artery.

### TECHNICAL FIELD

The present inventive concept relates to a method, a device, and a system for estimating a measure of cardiovascular health of a subject. The present inventive concept also relates to a computer program product for causing the method to be performed.

### BACKGROUND

Cardiovascular disease is a common problem. It is therefore of large interest to enable estimating cardiovascular health of subjects in a simple and robust manner.

Pulse wave velocity (PWV) is a measure of interest, which may provide a clinical measure of arterial stiffness and which may also be used for estimating blood pressure of a subject.

At present, a clinical gold standard measure is carotid-to-femoral PWV (PWV_{cf}), which may be estimated from a distance between two anatomic landmarks divided by a differential time interval measurement.

However, measurements at plural anatomic landmarks may require a fairly complicated system with plural sensors and the use of such a system requires specially trained operators.

It would therefore be desired to have a simple system which enables robust determination of PWV.

### SUMMARY

An objective of the present inventive concept is to provide robust estimation of a measure of cardiovascular health of a subject using a compact system. It is a particular object to enable estimation using a sensor for detecting pressure pulse wave propagation at a single location in an artery of the subject.

These and other objectives of the present inventive concept are at least partly met by the invention as defined in the independent claims. Preferred embodiments are set out in the dependent claims.

According to a first aspect, there is provided a method for estimating a measure of cardiovascular health of a subject, said method comprising: receiving time-based sequences of at least a first artery signal and a second artery signal, each representative of pressure pulse wave propagation in an artery of the subject, wherein the at least first and second artery signals represent pressure pulse wave propagation in positions displaced in relation to each other in a segment of the artery of the subject; fitting a first waveform and a second waveform to a portion of the time-based sequences of the at least first artery signal and second artery signal to form a first waveform and a second waveform of the first artery signal and a first waveform and a second waveform of the second artery signal, wherein the first waveforms of the first and second artery signals represent a forward propagating wave in the artery between the positions at which pressure pulse wave propagation is represented and the second waveforms of the first and second artery signals represent a backward propagating wave in the artery between the positions at which pressure pulse wave propagation is represented; and determining at least one parameter based on the fitting of the first waveform and the second waveform, wherein the at least one parameter comprises a forward velocity of the pressure pulse wave propagation as a representation of a local pulse wave velocity in the artery.

It is an insight of the present inventive concept that accurate determination of local pulse wave velocity in an artery cannot be based on following progress of a fiducial point in the pressure pulse wave as the pressure pulse wave propagates between positions in the artery. A forward pressure pulse wave in the artery is affected by backward propagating wave(s) due to reflections in the artery system, such as reflections from a branching in the artery system. Such backward propagating wave(s) will affect the fiducial point in the pressure pulse wave such that a position in time of the fiducial point will not be correspondingly represented at different positions in the artery. Hence, determination of local pulse wave velocity based on a time difference of a fiducial point of a pressure pulse wave between different positions in the artery will inevitably be incorrect due to displacement of the fiducial point based on backward propagating wave(s).

Thanks to the present inventive concept, it is possible to robustly determine a local pulse wave velocity of the subject. The present inventive concept performs a fitting of a first waveform and a second waveform to at least a first and a second artery signal, representing pulse wave propagation in different positions. This implies that the fitting takes into account both the first and the second artery signal when forming the first and the second waveforms. Thus, a spatial dimension (displaced positions for the first and the second artery signal) is taken into account, which ensures that the fitting of the first and second waveforms is based on information of progress of forward and backward propagating waves. Hence, respective contributions of the forward and backward propagating waves may be extracted from the combined pressure pulse wave.

Correctly splitting a pressure pulse wave into a forward propagating wave and a backward propagating wave based on a single artery signal is very difficult. Since the invention uses both the first and the second artery signal in order to fit the first and the second waveforms to both artery signals, a correct fitting of waveforms to represent the forward propagating wave and the backward propagating wave is enabled.

It should be realized that the fitting of the first waveform and the second waveform to the first and the second artery signals implies that corresponding waveforms are used for representing both the first and the second artery signals. The first waveform of the first artery signal thus has a similar or the same shape as the first waveform of the second artery signal and the first waveforms may be represented by similar or identical parameters for defining the waveforms. The first waveform of the second artery signal is displaced in time and position and possibly attenuated in relation to the first waveform of the first artery signal such that the amplitude of the first waveform is not the same for the first and the second artery signal. The same applies to the second waveforms of the first and the second artery signals.

Thanks to the fitting of the first and the second waveforms, a forward propagating wave and a backward propagating wave in the artery is determined. This allows accurate determination of the propagation of pressure pulse wave in the artery. The first and second waveforms may thus be used for determining a forward velocity of pressure pulse wave propagation in the artery. This could be used in itself as a measure of local pulse wave velocity in the artery providing a measure of cardiovascular health of the subject. However, it should be realized that further analysis of the first and second waveforms may be performed in order to determine other measures of the cardiovascular health based on parameters directly obtainable through the fitting of the first and the second waveforms or through further processing, such as through waveform analysis.

As used herein, the term “subject” for which a measure of cardiovascular health is estimated should be construed as a human being.

As used herein, the term “artery signal” should be construed as any signal representative of pressure pulse wave propagating in an artery. The artery signal may thus be acquired in many different manners, using a non-invasive sensor arranged outside a body of the subject, such as arranged on the skin of the subject in relation to the location in the artery. For example, the artery signal may be acquired by passive sensing, such as acquiring vibrations caused by pressure pulse wave propagation or by active sensing, such as transmitting a signal to the artery and detecting a response to the transmitted signal for detecting pressure pulse wave propagation. The artery signal could be a representation of the pressure or an influence of the pressure provided by the pressure pulse. However, the artery signal could alternatively be a representation of a speed of change of the pressure in the pressure pulse, such as could be acquired by an accelerometer.

The first artery signal and the second artery signal represent pressure pulse wave propagation in positions displaced in relation to each other in a segment of the artery. Thus, the first artery signal and the second artery signal are acquired from different positions within the artery. The first artery signal and the second artery signal represent pressure pulse wave propagation in the same segment of the artery, which implies that the first and second artery signals are acquired from positions close to each other. Hence, the first and second artery signals are not acquired from opposite sides of a branching of the artery system.

As used herein, the term “time-based sequence” should be construed as values arranged in a sequence representing a variation in time. The time-based sequence can represent a signal in a particular position. The time-based sequence could be an analog sequence, such that a signal that is continuous in time is represented by the time-based sequence. Alternatively, the time-based sequence could be a digital sequence, such that a sequence of discrete values is provided.

The fitting of the first and second waveforms could imply that the first and second waveforms are represented by parameters defining the shapes of the waveforms in order for the fitting to take place. Thus, parameters of the first and second waveforms may be determined through the fitting step. Thus, “determining at least one parameter based on the fitting” could imply that at least one parameter of the first and second waveforms is fetched from the fitting step (e.g. fetched from a register in a memory). For instance, the forward velocity of the pressure pulse wave propagation may be determined directly based on parameters of the fitting defining the first and second waveforms of the first and second artery signals. The determining of at least one parameter may also include determining a backward velocity of a reflected wave of the pressure pulse wave propagation, which backward velocity may also be determined directly based on parameters of the fitting defining the first and second waveforms of the first and second artery signals. However, the determining of the at least one parameter may alternatively imply that processing of parameters or other values obtained from the fitting step is performed.

The fitting of the first and the second waveforms may be performed by analytically processing the first and the second artery signals. Such analytical processing may include determination of fiducial point(s) in the first and second artery signals, based on which the first and second waveforms to be fitted to the artery signals. However, it should be understood that the first and the second artery signals need not necessarily be analytically processed. Rather, a machine-learned algorithm may be used, wherein the machine-learned algorithm may have been trained based on training data. Then, the machine-learned algorithm may perform the fitting without necessarily performing intermediate steps, such as determining fiducial point(s) of the first and second artery signals.

The first and second waveforms are fitted to a portion of the time-based sequences of the first and the second artery signals. This implies that the fitting may be performed in relation to e.g. a portion of a cardiac cycle. Hence, a representation of the local pulse wave velocity at a particular portion of the cardiac cycle may be determined and it is even possible to determine plural local pulse wave velocities for different portions of the time-based sequences by separately performing the fitting step for different portions. Hence, differences in local pulse wave velocities for different portions of the cardiac cycle may be determined allowing further analysis of a cardiovascular health of the subject.

The method determines a local pulse wave velocity representing a velocity of pressure pulse wave propagation locally in the segment of the artery at which the first and the second artery signals are acquired. This could be used for estimating local cardiovascular health, such as local arterial stiffness. However, the local pulse wave velocity could also be used as an indication of an overall cardiovascular health of the subject, such as a systemic stiffness of the arterial system.

The method is computer-implemented, such that the method may be performed by a processing unit of a computer. The processing unit may be arranged in a sensor or directly connected to the sensor that acquires the first and second artery signals. However, the processing unit may alternatively be arranged in a separate device, which may be arranged anywhere in relation to the sensor, such as even a processing unit arranged “in the cloud”. The processing unit may be configured to receive the time-based sequences of the first and second artery signals through a wired or wireless communication with a sensor that acquires the signals.

The processing unit may be implemented as a processing unit that executes a software program for performing the method. According to an alternative, the processing unit may be implemented as a dedicated hardware circuitry, such as an Application-Specific Integrated Circuit (ASIC).

According to an embodiment, each of the first artery signal and the second artery signal represents acceleration of the pressure pulse wave propagating in the artery of the subject.

The acceleration of the pressure pulse wave allows determining high-frequency components, such that high-frequency components are analyzed in the first and the second artery signal. The high-frequency components emphasize propagation of pressure pulse waves between positions close to each other such that robust fitting of a forward propagating wave and a backward propagating wave is facilitated.

It should be realized that the first artery signal and the second artery signal could be determined to represent an acceleration by a direct acceleration measurement by a sensor, e.g. using an accelerometer. However, according to an alternative, the first and second artery signals could be determined to represent acceleration by obtained directly or through one or more differentiations, i.e. actions of computing a derivative.

According to an embodiment, each of the first artery signal and the second artery signal is a second derivative of a distension waveform at respective positions in the segment of the artery.

The distension waveform may represent changes to a diameter of the artery as caused by pressure pulse wave and blood flow propagating through the artery. The distension waveform may be suitable for robustly determining pressure pulse wave propagation in the artery. Then, by computing second derivatives of acquired distension waveforms, the first and second artery signals may be robustly determined, while representing acceleration of pressure pulse wave propagation.

According to an embodiment, the method further comprises, before said fitting, extracting fiducial points in the time-based sequences of the at least first artery signal and second artery signal.

The extraction of fiducial points facilitates analytically determining the first and the second waveforms in the fitting step. These fiducial points may be used for determining end points of the portion of the time-based sequences, which is to be used for fitting the first and the second waveforms to the first and the second artery signals. Also, the fiducial points may be used for determining initial fitting parameters, which may be used as initial values in an iterative determination of the first and second waveforms.

The extraction of fiducial points may facilitate robust determination of the forward and backward propagating waves. Also, the use of fiducial points in determining the forward and backward propagating waves may imply that the representation of the pressure pulse wave propagation is physiologically comprehensible. This may make the estimated measures of cardiovascular health attractive to be used by a physician in making a diagnosis.

According to an embodiment, the extracting of fiducial points is achieved through at least one of: determining a local minimum in any one of the distension waveform or a first to fifth derivative of the distension waveform, determining a local maximum in any one of the distension waveform or a first to fifth derivative of the distension waveform, determining an inflection point in any one of the distension waveform or a first to fifth derivative of the distension waveform.

Determining of local minima, maxima, and inflection points can be robustly performed in analysis of a signal. Hence, such points may form suitable fiducial points to be used in analysis of acquired distension waveforms.

According to an embodiment, the method further comprises acquiring a first and a second distension waveform using a first and a second ultrasound sensor, and calculating a second derivative of the first distension waveform to form the first artery signal and calculating a second derivative of the second distension waveform to form the second artery signal.

An ultrasound sensor may be used for detecting the artery signal as a response to an ultrasound signal transmitted towards the artery. An artery signal obtained by an ultrasound sensor may clearly represent the pressure pulse wave propagation in the artery to allow robust detection of the pressure pulse wave.

The ultrasound sensor may be compact and arranged on a small carrier, such as a patch, that may be locally arranged in relation to the segment of the artery.

The ultrasound sensor may further be configured to be arranged in contact with skin of the subject. The ultrasound sensor may thus be placed in contact with the skin, without a need to pinch or clamp the skin of the subject for improving the relation between the sensor and the artery (as may be needed if a tonometer would be used for determining the artery signals, since the tonometer may need direct mechanical coupling with the artery).

This ensures that the ultrasound sensor may be comfortably worn so as to facilitate continuous monitoring of the subject wearing the ultrasound sensor for a long period of time.

According to another embodiment, the first and second artery signals are based on sensing by an optical sensor, a piezoelectric tonometer, a bioimpedance sensor or a radio frequency sensor.

Thus, the first and second artery signals need not necessarily be based on detection by ultrasound sensors, but other alternatives may be considered. For instance, sensors based on optical principles, such as a photoplethysmography sensor or an optical coherence tomography sensor may be used. The sensors may be used for detecting signals representing pressure pulse wave and/or blood flow propagating through the artery.

According to an embodiment, the first and the second distension waveforms are acquired by an array of ultrasound sensors configured to acquire distension waveforms from a carotid artery of the subject.

Using an array of ultrasound sensors allows arranging the first and the second ultrasound sensor in an array, which facilitates having a compact system for detecting waveforms from the subject. The array of ultrasound sensors could include a number of sensors, such that more than two artery signals could be easily obtained from a compact system. Having more than two artery signals implies that more than two artery signals are involved in analysis of pressure pulse wave propagation, which may be used for improving robustness of determining the forward and backward propagating waves.

It may be possible to obtain a strong signal of pressure pulse wave propagation in the carotid artery such that acquiring distension waveforms from a carotid artery allows accurate determination of the local pulse wave velocity.

Further, using the carotid artery enables determination of a central pulse wave velocity, i.e. a pulse wave velocity through large arteries of the subject.

According to an embodiment, receiving time-based sequences comprises receiving time-based sequences of at least the first artery signal, the second artery signal, and a third artery signal.

Thus, in addition to the first artery signal and the second artery signal, a third artery signal may also be received. The third artery signal may contribute to the fitting of the first waveform and the second waveform, such that the fitting of the waveforms may be improved by using further information of pressure pulse wave propagation in the artery of the subject.

Each of the first, second, and third artery signals may represent pressure pulse wave propagation at a unique position in the segment of the artery of the subject. Further, the first and the second waveforms may be fitted against each of the first, second and third artery signals, such that fitting is done in relation to three different positions in the artery of the subject. This implies that the fitting forms a first waveform and a second waveform also for the third artery signal.

The first, second and third artery signals may be acquired by an array of sensors. It should be realized that more than three artery signals may be used, such as eight, 16 or 32 artery signals.

When reference is made herein to first and second artery signals, it should be realized that further artery signals could be used, such as three, eight, 16 or 32 artery signals, unless explicitly stated otherwise.

According to an embodiment, each of said first waveform and said second waveform is a Gaussian waveform.

A Gaussian waveform is a suitable waveform for representing the forward and backward propagating waves such that a good fit may be obtained. The fitting of the first and second waveforms to the portion of the time-based sequences of the first and second artery signals may thus be pre-set to use a Gaussian waveform. However, it should be realized that other waveforms may be used instead as templates for the fitting step.

The same type of waveform may be used for the fitting of the first waveform and the second waveform, e.g. both the first and the second waveform may be Gaussian waveforms. Alternatively, different types of waveforms may be used for the fitting of the first waveform and the fitting of the second waveform.

According to an embodiment, the portion of the time-based sequences corresponds to a diastolic trough to systolic peak within a single heartbeat.

This portion of the time-based sequences of the first and the second artery signal may be suitable for determining a forward propagating wave and a backward propagating wave. In this portion, distinct characteristics of the pressure pulse wave propagation are apparent, such that the portion is suited for robust determination of the forward propagating wave and the backward propagating wave.

The diastolic trough corresponds to an absolute minimum of a distension waveform of a peripheral artery. This absolute minimum directly precedes the systolic foot, which is the maximum acceleration of the pressure pulse wave.

According to another embodiment, the portion of the time-based sequences corresponds to a dicrotic notch within a single heartbeat.

This portion of the time-based sequences of the first and the second artery signal may also be suitable for determining a forward propagating wave and a backward propagating wave. In this portion, distinct characteristics of the pressure pulse wave propagation are apparent, such that the portion is suited for robust determination of the forward propagating wave and the backward propagating wave.

However, it should be realized that any portion of the time-based sequences from diastole to diastole (i.e. a cardiac cycle) as seen in the artery could be used.

It should further be realized that plural fittings may be performed for different portions within a cardiac cycle. Pressure pulse wave propagation may exhibit different velocities for different portions of the cardiac cycle. Thus, by determining pulse wave velocities in the artery for different portions of the cardiac cycle further information of cardiovascular health of the subject may be obtained.

Also, it should be realized that fitting of the first waveform and the second waveform may be performed in a plurality of cardiac cycles. Thus, pulse wave velocities may be determined for several pulses such that a variation of pulse wave velocities amongst different pulses may be observed.

According to an embodiment, the at least one parameter is a parameter describing the first or the second waveform.

The first and the second waveforms may each be defined by a plurality of parameters. Hence, the fitting of the first and the second waveforms determines parameters that describes the first and the second waveform. The step of determining at least one parameter may thus comprise directly using a parameter that is determined in the fitting step.

This implies that direct use is made of the parameter(s) determined in the fitting step, such that the at least one parameter to be used in estimating a measure of cardiovascular health may be quickly and easily obtained.

However, according to an alternative, the determining of the at least one parameter comprises processing one or more of the parameters describing the first and the second waveforms for obtaining the at least one parameter comprising forward velocity of the pressure pulse wave propagation.

According to an embodiment, the fitting of the first waveform and the second waveform comprises iteratively changing a set of parameters for reducing an error between the first waveform and the second waveform and the portion of the time-based sequences of the at least first artery signal and second artery signal.

Thus, iterations may be used for improving the fitting of the first and the second waveforms. In each iteration, at least one parameter in a set of parameters describing the first and the second waveforms is changed for improving the fitting. The iterations aim to reduce the error between the first waveform and the second waveform and the portion of the time-based sequences of the first and second artery signals as much as possible.

The iterative changing of the set of parameters may proceed until the error is no longer reduced, such that the fitting is optimized.

Alternatively, the iterative changing of the set of parameters may proceed until the error has been reduced below a threshold value, such that it is considered that the fitting is sufficient.

According to yet another alternative, the iterative changing of the set of parameters may proceed until a maximum number of iterations have been performed.

The fitting may comprise checking whether any of the above alternative conditions for terminating the iterative changing is met and terminating the iterative changing if any of the conditions is met. However, it should be realized that the fitting need not check for all of the alternative conditions. Rather, in some embodiments, one or two of the conditions may be used for determining whether the iterative changing is to be terminated.

According to an embodiment, the method further comprises, during iterative changing of the set of parameters, determining a quality of fitting of the first waveform and the second waveform to the portion of the time-based sequences of the at least first artery signal and second artery signal.

The quality of fitting may be used for representing the error between the first waveform and the second waveform and the portion of the time-based sequences of the at least first artery signal and second artery signal. The quality of fitting may also be used as a measure of how well the first and the second waveforms fit to the portion of the time-based sequences of the at least first artery signal and second artery signal, such that the quality of fitting may be an indication of how reliable the determined parameters based on the fitting are. This may be useful in further assessment of the determined parameters.

According to an embodiment, the method further comprises normalizing an amplitude of the first and the second artery signal before said fitting of the first waveform and the second waveform to the portion of the time-based sequences of the at least first artery signal and second artery signal.

The normalizing of the amplitude is useful for improving robustness of the fitting of the first and second waveforms. For instance, normalizing may accommodate for potential discrepancies in acquisition for determining the first and the second artery signals.

According to another embodiment, the method further comprises detrending of the first and the second artery signal before said fitting of the first waveform and the second waveform to the portion of the time-based sequences of the at least first artery signal and second artery signal.

The detrending is useful for improving robustness of the fitting of the first and second waveforms. For instance, detrending may remove low-frequency wave components.

Both normalizing of the amplitude and detrending may ensure that fitting is performed with a higher weighting to temporal characteristics (i.e. how the pressure pulse wave propagates between the positions in the artery) compared to amplitude characteristics. This is useful for providing a better estimate of the local pulse wave velocity (which is a temporal characteristic).

According to an embodiment, the forward velocity of the pressure pulse wave propagation is used for estimating a stiffness of the segment of the artery of the subject.

Pulse wave velocity is a useful measure for assessing arterial stiffness. Hence, the forward velocity may be used for estimating the stiffness of the segment of the artery, which may be used as a measure of cardiovascular health of the subject.

According to an embodiment, the method further comprises estimating blood pressure of the subject based on the at least one parameter.

Blood pressure is a measure that is of high interest for generally assessing cardiovascular health of the subject. However, blood pressure is normally acquired using an inflatable cuff, which is inconvenient, and may require a trained operator for correctly determining the blood pressure. Also, the blood pressure may be affected by circumstances in which it is acquired, e.g. in a doctor's office, such that the acquired blood pressure does not accurately reflect the subject's health.

A correct estimation of pulse wave velocity may be used for determining a corresponding blood pressure. Hence, the method estimating blood pressure based on determined pulse wave velocity may advantageously provide an estimate of blood pressure using a system that may be worn for a long time and which may imply that the blood pressure is not increased by the circumstances in which it is measured. Also or alternatively, an estimation of a change of pulse wave velocity may be used for determining a corresponding change of blood pressure.

Further, as mentioned above, the pressure pulse wave propagation may exhibit different local pulse wave velocities in different portions of the cardiac cycle. Thus, corresponding different levels of blood pressure may be determined within the cardiac cycle, which may allow for further analysis of the cardiovascular health of the subject.

According to a second aspect, there is provided a computer program product comprising computer-readable instructions such that when executed on a processing unit the computer-readable instructions will cause the processing unit to perform the method according to the first aspect.

Effects and features of this second aspect are largely analogous to those described above in connection with the first aspect. Embodiments mentioned in relation to the first aspect are largely compatible with the second aspect.

The computer program product may implement the method in a processing unit, which may be a dedicated processing unit for performing the method or may be a general-purpose processing unit which may be able to perform the method based on the computer program product.

The computer program product may be provided on a tangible computer-readable medium provided with the computer-readable instructions, such as any computer-readable medium on which the computer-readable instructions may be stored.

According to a third aspect, there is provided a device for estimating a measure of cardiovascular health of a subject, said device comprising: a processing unit configured to: receive time-based sequences of at least a first artery signal and a second artery signal, each representative of pressure pulse wave propagation in an artery of the subject, wherein the at least first and second artery signals represent pressure pulse wave propagation in positions displaced in relation to each other in a segment of the artery of the subject; fit a first waveform and a second waveform to a portion of the time-based sequences of the at least first artery signal and second artery signal to form a first waveform and a second waveform of the first artery signal and a first waveform and a second waveform of the second artery signal, wherein the first waveforms of the first and second artery signals represent a forward propagating wave in the artery between the positions at which pressure pulse wave propagation is represented and the second waveforms of the first and second artery signals represent a backward propagating wave in the artery between the positions at which pressure pulse wave propagation is represented; and determine at least one parameter based on the fitting of the first waveform and the second waveform, wherein the at least one parameter comprises a forward velocity of the pressure pulse wave propagation as a representation of a local pulse wave velocity in the artery.

Effects and features of this third aspect are largely analogous to those described above in connection with the first and second aspects. Embodiments mentioned in relation to the first and second aspects are largely compatible with the third aspect.

According to a fourth aspect, there is provided a system for estimating a measure of cardiovascular health of a subject, said system comprising: the device according to the third aspect; and at least a first artery signal sensor and a second artery signal sensor, wherein the first artery signal sensor and the second artery signal sensor are configured to sense pressure pulse wave propagation in the artery of the subject in positions displaced in relation to each other in the segment of the artery of the subject for generating the first artery signal and the second artery signal, respectively.

Effects and features of this fourth aspect are largely analogous to those described above in connection with the first, second, and third aspects. Embodiments mentioned in relation to the first, second, and third aspects are largely compatible with the fourth aspect.

The system may enable determining of the local pulse wave velocity using wearable device(s) that may be worn by the subject for a prolonged period of time to estimate the local pulse wave velocity while minimally affecting daily life of the subject.

The processing unit may be arranged integrated with the artery signal sensors such that the processing unit may receive the first and second artery signals through communication within an integrated unit for further processing the signals and determining the local pulse wave velocity of the subject.

The first and second artery signals may be directly generated by the first and second artery signal sensors, respectively. However, the first and second artery signals may alternatively be generated through signal processing of signals acquired by the first and second artery signals. Such processing may include e.g. computation of a (second) derivative of the acquired signal. The signal processing to generate the first and second artery signals may be performed by a separate signal processing unit, such as a dedicated integrated circuit or may be performed within (a separate part or module of) the processing unit which also performs fitting of the first and second waveforms and determining of the at least one parameter.

The processing unit may alternatively be arranged in a separate housing, which may or may not be worn by the subject. The processing unit may receive signals from the first and the second artery signal sensors by wired or wireless communication.

The processing unit being provided in a wearable device may provide that the subject may be presented with the measure of the cardiovascular health in the wearable device in real time. Thus, the subject may continuously have information of the cardiovascular health available. The processing unit may be arranged in a device that the subject may anyway wear, such as in a smartwatch.

Alternatively, the processing unit may be provided in a unit that may not necessarily be worn by the subject, but which may be available for short-range communication with the artery signal sensor, such as the processing unit being arranged in a smartphone.

As a further alternative, the processing unit may be provided anywhere, such as “in the cloud”. The processing unit may communicate with the artery signal sensors through a computer network, such as the Internet, enabling the processing unit to be arranged anywhere in relation to the artery signal sensors. The processing unit may communicate results of the determination of the local pulse wave velocity of the subject back to the subject to enable presentation to the subject, e.g. communicating to a smartphone, to a wearable device, or to the artery signal sensors.

According to an embodiment, the at least first artery signal sensor and second artery signal sensor are arranged in an array of ultrasound sensors configured to acquire distension waveforms from a carotid artery of the subject.

The array of ultrasound sensors may be compact and arranged on a small carrier, such as a patch, that may be locally arranged in relation to the segment of the artery. This ensures that the ultrasound sensors may be comfortably worn so as to facilitate continuous monitoring of the subject wearing the ultrasound sensors for a long period of time.

It may be possible to obtain a strong signal of pressure pulse wave propagation in the carotid artery such that acquiring distension waveforms from a carotid artery allows accurate determination of the local pulse wave velocity.

According to another embodiment, the first and second artery signal sensors are optical sensors, piezoelectric tonometers, bioimpedance sensors or radio frequency sensors.

Thus, the first and second artery signals need not necessarily be based on detection by ultrasound sensors, but other alternatives may be considered. For instance, sensors based on optical principles, such as a photoplethysmography sensor or an optical coherence tomography sensor may be used. The sensors may be used for detecting signals representing pressure pulse wave and/or blood flow propagating through the artery.

### BRIEF DESCRIPTION OF THE DRAWINGS

The above, as well as additional objects, features, and advantages of the present inventive concept, will be better understood through the following illustrative and non-limiting detailed description, with reference to the appended drawings. In the drawings like reference numerals will be used for like elements unless stated otherwise.

FIG. 1 shows graphs illustrating a problem of correctly determining pulse wave velocity in an artery.

FIG. 2 is a flowchart of a method according to an embodiment.

FIG. 3 is a schematic view of a system according to an embodiment.

FIG. 4 shows graphs illustrating fitting of a double Gaussian propagation model.

### DETAILED DESCRIPTION

Pulse wave velocity (PWV) is a useful measure for assessing cardiovascular health of a subject. In principle, a local PWV could be determined by determining a time difference of a pressure pulse wave between different positions along an artery. Looking at the time difference of a characteristic of the pressure pulse wave between the positions, the time difference may be determined. Then, local PWV can be determined by dividing a distance between the positions by the determined time difference.

However, a pressure pulse wave observed in a position of an artery is based on confluence of forward propagating waves and backward propagating waves due to reflections in the artery system. Thus, the characteristic of the pressure pulse wave is affected by backward propagating waves, such that local PWV estimations will be biased by the confluence of forward and backward propagating waves.

The confluence of forward and backward propagating waves varies to different extents between human beings. This further means that there is a need to handle this confluence of forward and backward propagating waves in order to correctly determine the local PWV.

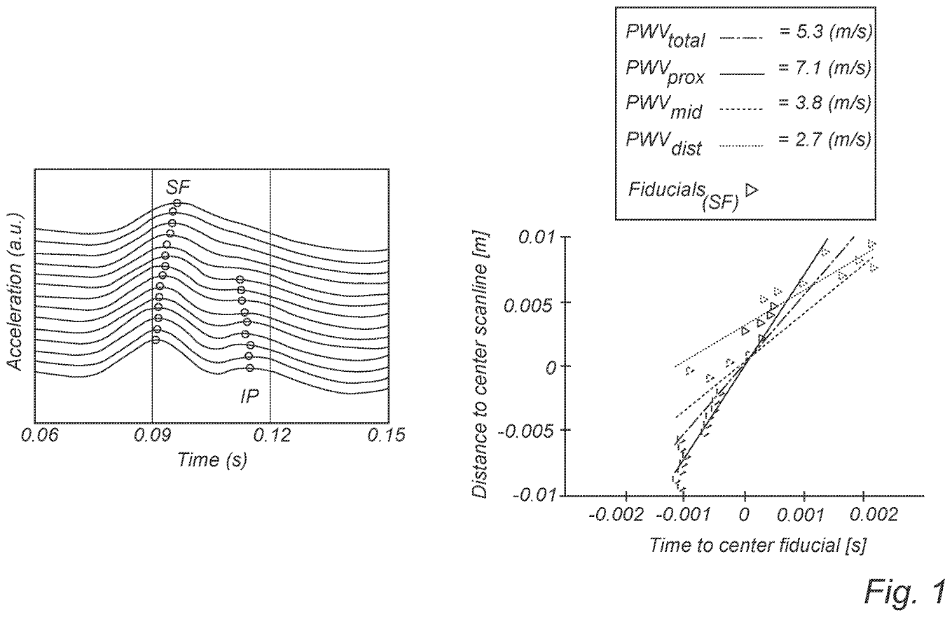

In FIG. 1, an acceleration of a pressure pulse wave as measured in several different positions along a carotid artery is shown in the left-hand graph. The waveforms show acceleration in a portion of the cardiac cycle between a diastolic trough and systolic peak. A first fiducial point corresponding to systolic foot is marked in the waveforms (circles denoted SF). A second fiducial point corresponding to an inflection point in the waveforms is marked in the waveforms (circles denoted IP). The inflection point is due to a backward propagating wave in the artery.

As can be seen, the first fiducial point appears first in time in a first waveform (bottom curve in the left-hand graph of FIG. 1) and arrives later in time as the pressure pulse wave propagates towards a distal part of the artery (further away from the heart). The second fiducial point appears first in time in a center waveform (center curve in the left-hand graph of FIG. 1) and arrives later in time as the backward propagating wave propagates towards a proximal part of the artery (closer to the heart). The second fiducial point cannot be clearly determined in the waveforms of the most distal parts of the artery.

FIG. 1 illustrates that the backward propagating wave affects the position in time of the first fiducial point compared to propagation of the forward propagating wave (and vice versa with regard to the second fiducial point). In the right-hand graph of FIG. 1, a position in the artery of the first fiducial point is plotted against time. The position in the artery is presented in terms of a distance in relation to a center scanline (a sensor arranged in a center of an array of sensors for acquiring the waveforms of the left-hand graph). The local PWV in the artery can be determined based on a curve in the right-hand graph fitted to the extracted first fiducial points. As illustrated in the right-hand graph of FIG. 1, different curves may be defined depending on which waveforms are taken into account when fitting the curve to the first fiducial points. A total (average) PWV taken all the first fiducial points into account is 5.3 m/s. However, an apparent proximal PWV taking only the waveforms of the most proximal positions in the artery into account is 7.1 m/s, whereas an apparent distal PWV taking only the waveforms of the most distal positions in the artery into account is 2.7 m/s and an apparent central PWV taking only the waveforms of the center positions between the proximal and distal positions into account is 3.8 m/s. Hence, it is clear that the local PWV cannot be reliably determined by merely assessing the propagation of the first fiducial point.

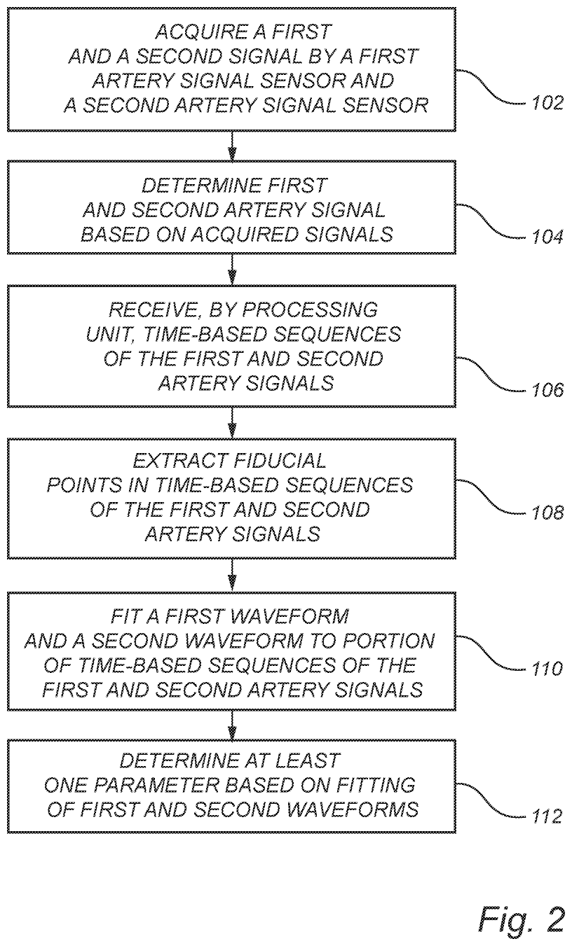

Referring now to FIG. 2, a method according to an embodiment of the present inventive concept will be described. The method intends to provide a robust determination of the local PWV in a segment of the artery of a subject based on analyzing both forward and backward propagating waves.

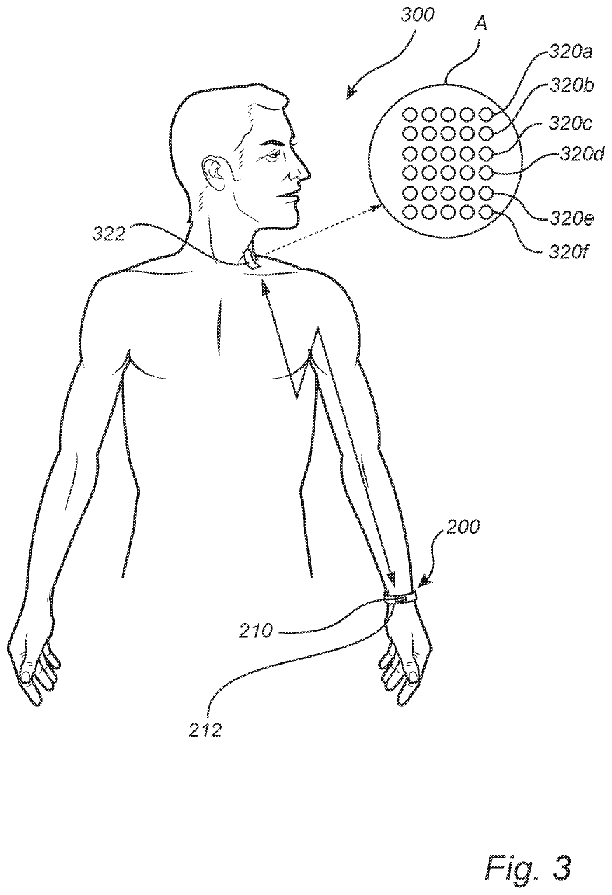

In the description of the method below, reference will also be made to a system 300 for performing the method, which is shown in FIG. 3. The system 300 comprises a device 200, which comprises a processing unit 210 for performing computer-implemented steps of the method.

The method may be a computer-implemented method of analyzing acquired information representing the pressure pulse wave propagation in a segment of the artery. Thus, in some embodiments, the method may consist only of steps for processing signals, which may be purely performed by the processing unit 210.

The processing unit 210 may be arranged arranged integrated with artery signal sensors 320a-320f of the system 300 such that the processing unit 210 may receive information representing the pressure pulse wave propagation in a segment of the artery through communication within a single physical housing for further processing the signals and determining the local PWV of the subject. The single physical housing may be designed as a wearable, since the artery signal sensors 320a-320f may need to be arranged in contact with skin of the subject in order to acquire signals representing the pressure pulse wave propagation in the segment of the artery.

The processing unit 210 may alternatively be arranged in a separate housing 212, which may or may not be worn by the subject. The processing unit 210 may receive signals from the artery signal sensors 320a-320f by wired or wireless communication.

The processing unit 210 being provided in a wearable device may provide that the subject may be presented with the measure of the cardiovascular health in the wearable device in real time. Thus, the subject may continuously have information of the cardiovascular health available. The processing unit 210 may be arranged in a device 200 that the subject may anyway wear, such as in a smartwatch.

Alternatively, the processing unit 210 may be provided in a unit that may not necessarily be worn by the subject, but which may be available for short-range communication with the artery signal sensors 320a-320f, such as the processing unit 210 being arranged in a smartphone.

As a further alternative, the processing unit 210 may be provided anywhere, such as “in the cloud”. The processing unit 210 may communicate with the artery signal sensors 320a-320f through a computer network, such as the Internet, enabling the processing unit 210 to be arranged anywhere in relation to the artery signal sensors 320a-320f. The processing unit 210 may communicate results of the determination of the local PWV of the subject back to the subject to enable presentation to the subject, e.g. communicating to a smartphone, to a wearable device, or to the artery signal sensors 320a-320f.

As yet another alternative, the processing unit 210 may be distributed such that part of the processing is performed in one physical location and other parts are performed in another physical location.

In case the processing unit 210 is integrated with or linked to the artery signal sensors 320a-320f for acquiring the information in the system 300, the method performed by the system can also include steps for acquiring the information representing the pressure pulse wave propagation. Thus, before computer-implemented processing of signals, the method may comprise acquiring 102 of a first and a second signal by a first artery signal sensor 320a and a second artery signal sensor 320b, the first and the second signals representing the pressure pulse wave propagation.

According to an embodiment, the first and second signals may be acquired as raw ultrasound radio frequency signals. The raw signals may be processed in order to represent a first distension waveform and a second distension waveform. Each of the first and second distension waveforms may suitably be acquired by first and second ultrasound sensors 320a, 320b from a carotid artery. Each of the first and second ultrasound sensors 320a, 320b may in fact comprise a set of ultrasound elements, such that the information acquired by the sets of ultrasound elements may be processed to form the first and second distension waveforms, respectively.

The distension waveforms may represent changes to a diameter of the artery as caused by pressure pulse wave and blood flow propagating through the artery. The distension waveforms may be suitable for robustly determining pressure pulse wave propagation in the artery and the ultrasound sensors 320a-320f may be configured to acquire distension waveforms.

It may be possible to obtain a strong signal of pressure pulse wave propagation in the carotid artery such that acquiring distension waveforms from a carotid artery allows accurate determination of the local PWV. Further, using the carotid artery enables determination of a central pulse wave velocity, i.e. a pulse wave velocity through large arteries of the subject.

The method may further comprise determining 104 first and second artery signals based on the first and second signals acquired by the first and second artery signal sensors 320a, 320b, respectively. For instance, each of the first and second distension waveforms may be processed to calculate a second derivative of the respective waveforms so as to form the first and second artery signals, respectively. Thus, each of the first artery signal and the second artery signal may represent acceleration of the pressure pulse wave propagating in the artery of the subject.

The first and second artery signals may alternatively be directly generated by the first and second artery signal sensors 320a, 320b, respectively. Thus, the first and second artery signals may directly acquire signals representing acceleration of the pressure pulse wave propagating in the artery of the subject, e.g. by the first and second artery signals being accelerometers.

The method further comprises receiving 106, by the processing unit 210, time-based sequences of at least the first artery signal and the second artery signal. Each of the first and the second artery signal is thus representative of pressure pulse wave propagation in the artery of the subject. Further, the at least first and second artery signals represent pressure pulse wave propagation in positions displaced in relation to each other in the segment of the artery of the subject.

It should be realized that the processing unit 210 may receive more than two artery signals, such that the processing unit 210 may receive time-based sequences of at least the first artery signal, the second artery signal and a third artery signal, and optionally time-based sequences of even further artery signals, such as receiving a total of eight, 16, or 32 artery signals. Each artery signal represents the pressure pulse wave propagation in a unique position of the segment of the artery.

The time-based sequences could be analog sequence, such that artery signals that are continuous in time are represented by the time-based sequences. Alternatively, the time-based sequences could be digital sequences, such that a sequence of discrete values is provided. If the processing unit 210 is configured to receive analog sequences, the processing unit 210 may first perform analog-to-digital conversion in order to allow digital processing of the artery signals.

The method further comprises processing by the processing unit 210 of the received time-based sequences of at least the first and second artery signals. Details of the processing will now be described.

The method may comprise extracting 108 fiducial points in the time-based sequences of the first and second artery signals.

The extracted fiducial points may be used for determining end points of a portion of the time-based sequences to be analyzed, wherein the portion may define a part of the cardiac cycle from diastole to diastole as seen in the artery or the portion may define an entire cardiac cycle.

The extracted fiducial points may also define characteristics in the first and second artery signals, which may be used in determining representations of forward and backward propagating waves in the first and second artery signals.

The extraction of fiducial points may facilitate robust determination of the forward and backward propagating waves. Also, the use of fiducial points in determining the forward and backward propagating waves may imply that the representation of the pressure pulse wave propagation is physiologically comprehensible. This may make the estimated measures of cardiovascular health based on the method attractive to be used by a physician in making a diagnosis. However, it should be realized that extracting fiducial points may not be necessary. If a machine learning method is trained to analyze time-based sequences of the first and second artery signals, the machine-learned method may determine representations of forward and backward propagating waves without first extracting fiducial points in the time-based sequences of the first and second artery signals.

The extracting of fiducial points may be achieved through at least one of: determining a local minimum in any one of the distension waveform or a first to fifth derivative of the distension waveform, determining a local maximum in any one of the distension waveform or a first to fifth derivative of the distension waveform, determining an inflection point in any one of the distension waveform or a first to fifth derivative of the distension waveform.

According to an embodiment, an upstroke of the distension waveform may be determined by determining a maximum in the first derivative of the distension waveform. Then, a second derivative of the distension waveform may be searched for maxima, preceding and succeeding to the determined maximum in the first derivative of the distension waveform. The preceding maximum in the second derivative defines the systolic foot and the succeeding maximum defines the dicrotic notch.

Both a complex of the time-based sequence around the systolic foot and a complex of the time-based sequence around the dicrotic notch provides information of a predominantly forward directed wave (a forward propagating wave) followed by an early reflected wave (a backward propagating wave).

Inflection points of the reflected wave may be extracted as fiducial points by detecting succeeding maxima (in relation to the maxima defining the systolic foot and the dicrotic notch, respectively) by zero crossings in a third derivative of the distension waveform or, with progression confluence of the forward directed wave and the reflected wave, as turning points by zero crossings in a fourth derivative of the distension waveform.

The portion of the time-based sequences to be analyzed may be selected as a complex around the systolic foot corresponding to a diastolic trough to a systolic peak within a single cardiac cycle. The portion of the time-based sequences to be analyzed may alternatively be selected as a complex corresponding to the dicrotic notch within a single cardiac cycle. It should be realized that each of these portions may be analyzed to allow determining local PWVs within different portions of the cardiac cycle.

Each of the artery signals may be normalized in amplitude to accommodate for potential discrepancies between the artery signal sensors 320a-320f.

Each of the artery signals may further be detrended to remove low-frequency wave components.

Both normalizing of the amplitude and detrending may ensure that the following analysis of the artery signals is performed with a high weight to temporal characteristics (i.e. how the pressure pulse wave propagates between the positions in the artery) compared to amplitude characteristics. This is useful for providing a better estimate of the local PWV.

The method further comprises fitting 110 a first waveform and a second waveform to the portion of the time-based sequences of the at least first artery signal and second artery signal to form a first waveform and a second waveform of the first artery signal and a first waveform and a second waveform of the second artery signal, wherein the first waveforms of the first and second artery signals represent a forward propagating wave in the artery between the positions at which pressure pulse wave propagation is represented and the second waveforms of the first and second artery signals represent a backward propagating wave in the artery between the positions at which pressure pulse wave propagation is represented.

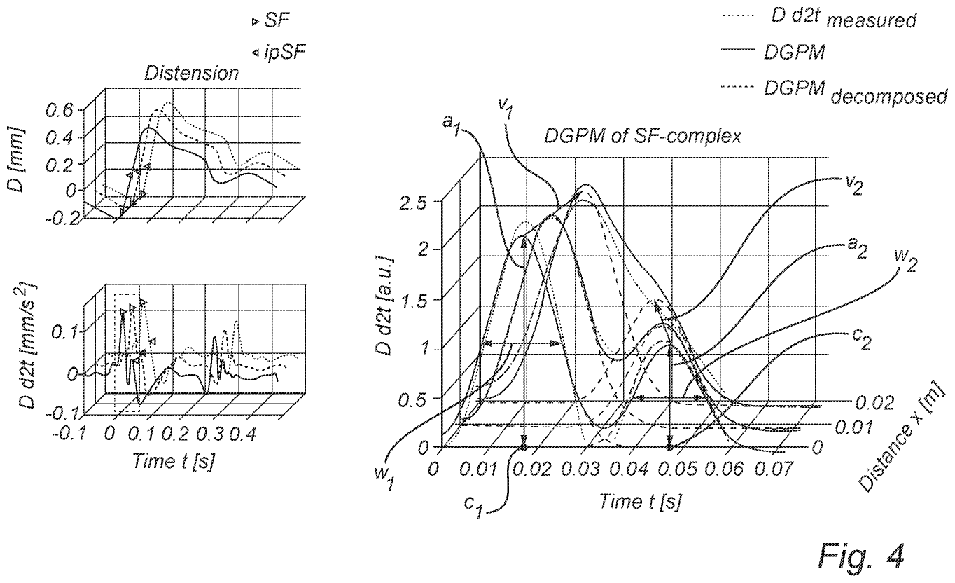

Referring now to FIG. 4, the fitting may use Gaussian waveforms for representing the first and second waveforms. However, it should be realized that use of other types of waveforms is possible.

When using Gaussian waveforms, the fitting is performed to generate a Double Gaussian Propagation Model (DPGM) for representing the portion of the time-based sequences.

The DPGM comprises two Gaussian waveforms to respectively fit the forward and backward propagating waves within the second derivative of the distension waveforms

$$( \frac{{d}^{2} D}{{dt}^{2}} ) .$$

The DPGM is defined as:

$$\frac{{d}^{2} D}{d {t}^{2}} ( t , x ) = {a}_{1} {e}^{{( \frac{t - ( {c}_{1} + \frac{x}{{v}_{1}} )}{{w}_{1}} )}^{2}} + {a}_{2} {e}^{{( \frac{t - ( {c}_{2} + \frac{x}{{v}_{2}} )}{{w}_{2}} )}^{2}} ,$$

wherein t denotes time [s], x denotes a distance [m] along the segment in the artery, and 8 parameters are used for modelling the first and second waveforms (see FIG. 4), namely a₁ and a₂ denoting the amplitude [arbitrary unit] of the first and second waveforms, respectively, c₁ and c₂ denoting a centroid [s] of the first and second waveforms, respectively, w₁ and w₂ denoting a width [s] of the first and second waveforms, respectively, and v₁ and v₂ denoting a velocity [m/s] of the forward and backward propagating waves represented by the first and second waveforms, respectively.

FIG. 4 shows in the top left-hand graph three different distension waveforms for three different positions in the artery. FIG. 4 further shows in the bottom left-hand graph three corresponding artery signals being second derivatives of the distension waveforms (distension acceleration waveforms). Fiducial points corresponding to the systolic foot (SF) and systolic foot inflection point (ipSF) are indicated in the top and bottom left-hand graphs. FIG. 4 further shows in the right-hand graph fitting of the DPGM to the complex around the systolic foot. The right-hand graph shows the distension acceleration waveforms (dotted lines) as well as the corresponding combined waveforms of the DPGM (solid lines) and the decomposed first and second waveforms of the DPGM (dashed lines). Further, the parameters are used for modelling the first and second waveforms are also indicated in the right-hand graph of FIG. 4.

The fitting of the first waveform and the second waveform may comprise iteratively changing the set of parameters for reducing an error between the first waveform and the second waveform and the portion of the time-based sequences of the at least first artery signal and second artery signal.

The iterations may be performed in relation to a root mean squared error (RMSE_{DPGM}) between measured waveforms (distension acceleration waveforms) and the DPGM. Initial values for the iterative fitting may be assessed from the extracted fiducial points, SF and ipSF. For instance, initial values of a₁, a₂, c₁, and c₂ may be based on respective amplitude and timing of the SF and ipSF features; w₁ and w₂ may be based on half an interval between the SF and ipSF features and v₁ and v₂ may be based on respective spatiotemporal gradient from most proximal to most distal waveforms.

The iterative fitting may proceed until the error is no longer reduced, such that the fitting is optimized. Alternatively, the iterative fitting may proceed until the error has been reduced below a threshold value, such that it is considered that the fitting is sufficient. According to yet another alternative, the iterative fitting may proceed until a maximum number of iterations have been performed.

The fitting may comprise checking whether any of the above alternative conditions for terminating the iterative fitting is met and terminating the iterative fitting if any of the conditions is met. However, it should be realized that the fitting need not check for all of the alternative conditions. Rather, in some embodiments, one or two of the conditions may be used for determining whether the iterative changing is to be terminated.

Quality of fitting (QoF) may be assessed during the iterative fitting and/or after the iterative fitting is terminated. The QoF may provide an indication of how well the DPGM is able to model the artery signals, which may be used for determining whether parameters determined based on the DPGM are reliable.

QoF may be assessed as percentage of the artery signals being accounted by the DPGM relative to a mean amplitude of the artery signals:

$$Q o F = 1 - \frac{R M S {E}_{D P G M}}{R M S {E}_{μ}} ,$$

wherein RMSE_{μ} is a root mean squared error between the measured waveforms (distension acceleration waveforms) and a mean value of the measured waveforms.

FIG. 4 illustrates use of three artery signals in fitting of the DPGM. It should be realized that at least two artery signals should be used, but many more than three artery signals could be used. However, for enhanced visibility in FIG. 4, only three artery signals are shown.

Referring again to FIG. 2, the method further comprises determining 112 at least one parameter based on the fitting of the first waveform and the second waveform. The at least one parameter may be determined by directly using one or more of the parameters defining the waveforms in the DPGM. Thus, the at least one parameter is a parameter describing the first or the second waveform. Alternatively or additionally, the at least one parameter may be determined by further processing the parameters defining the waveforms in the DPGM or other features that may be extracted from the DPGM.

The at least one parameter comprises a forward velocity of the pressure pulse wave propagation as a representation of a local PWV in the artery. The forward velocity may for instance be estimated using the parameter v₁ of the DPGM directly.

The at least one parameter may be further used to determine further measures of the cardiovascular health of the subject. According to an embodiment, the forward velocity of the pressure pulse wave propagation is used for estimating a stiffness of the segment of the artery of the subject.

According to another embodiment, the method further comprises estimating blood pressure of the subject based on the at least one parameter.

A correct estimation of PWV may be used for determining a corresponding blood pressure. As mentioned above, the pressure pulse wave propagation may exhibit different local PWVs in different portions of the cardiac cycle. Thus, corresponding different levels of blood pressure may be determined within the cardiac cycle, which may allow for further analysis of the cardiovascular health of the subject.

Referring now to FIG. 3, a device 200 and a system 300 for estimating a measure of cardiovascular health of the subject according to an embodiment will be described.

The device 200 may be configured to perform the computer-implemented signal processing steps of the method described above. Thus, the device 200 comprises a processing unit 210 which is configured to perform the signal processing steps, such as steps 106, 108, 110, and 112 described above.

The processing unit 210 may be implemented as any special-purpose or general-purpose processing unit, such as a central processing unit (CPU) that executes a software program for performing the signal processing steps. According to an alternative, the processing unit 210 may be implemented as a dedicated hardware circuitry, such as an Application-Specific Integrated Circuit (ASIC).

The device 200 may be configured as a wearable device, as shown in FIG. 3, such that the device 200 may be worn by the subject. Thus, the device 200 may be configured to be attached to a body part or to be arranged around a body part of the subject. For instance, as shown in FIG. 3, the device 200 may be configured to be worn around an arm of the subject, such as being implemented in a smartwatch.

The device 200 may thus comprise a housing 212, which carries the processing unit 210 and which is configured for attachment or arrangement of the device 200 at or around a body part of the subject.

The device 200 may further comprise a communication unit for communicating with entities external to the housing 212 of the device 200. The communication unit may be configured for wired or wireless communication with external entities.

The system 300 includes the device 200. The system 300 further comprises the artery signal sensors 320a-320f (illustrated in enlargement A of FIG. 3) for acquiring signals such that the artery signals described above can be generated.

According to an embodiment, the artery signal sensors 320a-320f are arranged in an array of ultrasound sensors configured to acquire distension waveforms from a carotid artery of the subject.

The array of ultrasound sensors 320a-320f may be compact and arranged on a small carrier, such as a patch 322, that may be locally arranged in relation to the segment of the artery. This ensures that the ultrasound sensors 320a-320f may be comfortably worn so as to facilitate continuous monitoring of the subject wearing the ultrasound sensors 320a-320f for a long period of time.

It may be possible to obtain a strong signal of pressure pulse wave propagation in the carotid artery such that acquiring distension waveforms from a carotid artery allows accurate determination of the local PWV.

However, it should be realized that ultrasound sensors are not necessarily used. According to another embodiment, the artery signal sensors are optical sensors, piezoelectric tonometers, bioimpedance sensors or radio frequency sensors.

Thus, the first and second artery signals need not necessarily be based on detection by ultrasound sensors, but other alternatives may be considered. For instance, sensors based on optical principles, such as a photoplethysmography sensor or an optical coherence tomography sensor may be used. The sensors may be used for detecting signals representing pressure pulse wave and/or blood flow propagating through the artery.

The artery signal sensors 320a-320f may further be associated with a communication unit for communication with the communication unit associated with the processing unit 210. In this way, the artery signals may be transferred from the artery signal sensors 320a-320f to the processing unit 210 for the signal processing to be performed therein.

In the above the inventive concept has mainly been described with reference to a limited number of examples. However, as is readily appreciated by a person skilled in the art, other examples than the ones disclosed above are equally possible within the scope of the inventive concept, as defined by the appended claims.

1. A method for estimating a measure of cardiovascular health of a subject, said method comprising:
receiving time-based sequences of at least a first artery signal and a second artery signal, each representative of pressure pulse wave propagation in an artery of the subject, wherein the at least first and second artery signals represent pressure pulse wave propagation in positions displaced in relation to each other in a segment of the artery of the subject;
fitting a first waveform and a second waveform to a portion of the time-based sequences of the at least first artery signal and second artery signal to form a first waveform and a second waveform of the first artery signal and a first waveform and a second waveform of the second artery signal, wherein the first waveforms of the first and second artery signals represent a forward propagating wave in the artery between the positions at which pressure pulse wave propagation is represented and the second waveforms of the first and second artery signals represent a backward propagating wave in the artery between the positions at which pressure pulse wave propagation is represented; and
determining at least one parameter based on the fitting of the first waveform and the second waveform, wherein the at least one parameter comprises a forward velocity of the pressure pulse wave propagation as a representation of a local pulse wave velocity in the artery.

2. The method according to claim 1, wherein each of the first artery signal and the second artery signal represents acceleration of the pressure pulse wave propagating in the artery of the subject.

3. The method according to claim 1, wherein each of the first artery signal and the second artery signal is a second derivative of a distension waveform at respective positions in the segment of the artery.

4. The method according to claim 1, further comprising, before said fitting, extracting fiducial points in the time-based sequences of the at least first artery signal and second artery signal.

5. The method according to claim 1, further comprising acquiring a first and a second distension waveform using a first and a second ultrasound sensor, and calculating a second derivative of the first distension waveform to form the first artery signal and calculating a second derivative of the second distension waveform to form the second artery signal.

6. The method according to claim 5, wherein the first and the second distension waveforms are acquired by an array of ultrasound sensors configured to acquire distension waveforms from a carotid artery of the subject.

7. The method according to claim 1, wherein receiving time-based sequences comprises receiving time-based sequences of at least the first artery signal, the second artery signal, and a third artery signal.

8. The method according to claim 1, wherein each of said first waveform and said second waveform is a Gaussian waveform.

9. The method according to claim 1, wherein the portion of the time-based sequences corresponds to a diastolic trough to systolic peak within a single heartbeat.

10. The method according to claim 1, wherein the portion of the time-based sequences corresponds to a dicrotic notch within a single heartbeat.

11. The method according to claim 1, wherein the at least one parameter is a parameter describing the first or the second waveform.

12. The method according to claim 1, wherein the fitting of the first waveform and the second waveform comprises iteratively changing a set of parameters for reducing an error between the first waveform and the second waveform and the portion of the time-based sequences of the at least first artery signal and second artery signal.

13. The method according to claim 12, further comprising, during iterative changing of the set of parameters, determining a quality of fitting of the first waveform and the second waveform to the portion of the time-based sequences of the at least first artery signal and second artery signal.

14. The method according to claim 1, further comprising normalizing an amplitude of the first and the second artery signal before said fitting of the first waveform and the second waveform to the portion of the time-based sequences of the at least first artery signal and second artery signal.

15. The method according to claim 1, wherein the forward velocity of the pressure pulse wave propagation is used for estimating a stiffness of the segment of the artery of the subject.

16. The method according to claim 1, further comprising estimating blood pressure of the subject based on the at least one parameter.

17. A computer program product comprising computer-readable instructions such that when executed on a processing unit the computer-readable instructions will cause the processing unit to perform the method according to claim 1.

18. A device for estimating a measure of cardiovascular health of a subject, said device comprising:
a processing unit configured to:
receive time-based sequences of at least a first artery signal and a second artery signal, each representative of pressure pulse wave propagation in an artery of the subject, wherein the at least first and second artery signals represent pressure pulse wave propagation in positions displaced in relation to each other in a segment of the artery of the subject;
fit a first waveform and a second waveform to a portion of the time-based sequences of the at least first artery signal and second artery signal to form a first waveform and a second waveform of the first artery signal and a first waveform and a second waveform of the second artery signal, wherein the first waveforms of the first and second artery signals represent a forward propagating wave in the artery between the positions at which pressure pulse wave propagation is represented and the second waveforms of the first and second artery signals represent a backward propagating wave in the artery between the positions at which pressure pulse wave propagation is represented; and
determine at least one parameter based on the fitting of the first waveform and the second waveform, wherein the at least one parameter comprises a forward velocity of the pressure pulse wave propagation as a representation of a local pulse wave velocity in the artery.

19. A system for estimating a measure of cardiovascular health of a subject, said system comprising:
the device according to claim 18; and
at least a first artery signal sensor and a second artery signal sensor, wherein the first artery signal sensor and the second artery signal sensor are configured to sense pressure pulse wave propagation in the artery of the subject in positions displaced in relation to each other in the segment of the artery of the subject for generating the first artery signal and the second artery signal, respectively.

20. The system according to claim 19, wherein the at least first artery signal sensor and second artery signal sensor are arranged in an array of ultrasound sensors configured to acquire distension waveforms from a carotid artery of the subject.
